(19) United States
(12) Patent Application Publication
(10) Pub. No.: US 2026/0137278 A1
(43) Pub. Date: May 21, 2026

(54) ULTRASONIC TONOMETER AND OPHTHALMIC ULTRASONIC ACTUATOR

(71) Applicant: NIDEK CO.,LTD., Gamagori-shi (JP)

(72) Inventor: Tsutomu UEMURA, Gamagori-shi (JP)

(21) Appl. No.: 19/449,421

(22) Filed: Jan. 15, 2026

Related U.S. Application Data
(63) Continuation of application No. PCT/JP2024/025420, filed on Jul. 16, 2024.

(30) Foreign Application Priority Data
Jul. 19, 2023 (JP) 2023-117773

(51) Int. Cl. A61B 3/16

(52) U.S. Cl. CPC A61B 3/165

(57) ABSTRACT

An ultrasonic tonometer includes an ultrasonic actuator having an ultrasonic element and configured to irradiate the subject eye with ultrasound generated by the ultrasonic element. The ultrasonic actuator includes: a sonotrode propagating ultrasound generated by the ultrasonic element into air; a flexural vibration portion disposed on a distal side of the sonotrode in an axial direction of the ultrasonic actuator; and a reflecting portion covering, with a gap with the flexural vibration portion, at least a part of the flexural vibration portion in a circumferential direction.

### CROSS REFERENCE TO RELATED APPLICATION

This application is a continuation application of International Patent Application No. PCT/JP2024/025420 filed on Jul. 16, 2024, which designated the U.S. and claims the benefit of priority from Japanese Patent Application No. 2023-117773 filed on Jul. 19, 2023. The entire disclosure of all of the above application is incorporated herein by reference.

### TECHNICAL FIELD

The present disclosure relates to an ultrasonic tonometer that measures the intraocular pressure of a subject eye using ultrasound, and to an ophthalmic ultrasonic actuator used in an ophthalmic apparatus such as an ultrasonic tonometer.

### BACKGROUND

Among noncontact tonometers, air-puff tonometers have been known. An air-puff tonometer measures intraocular pressure based on the deformation state of the cornea when air is jetted to the cornea and on the air pressure applied to the cornea.

Moreover, as a noncontact tonometer, an ultrasonic tonometer that measures intraocular pressure using ultrasound has been proposed. For example, the ultrasonic tonometer disclosed in Patent Document 1 (JP H05-253190 A) measures intraocular pressure based on the deformation state of the cornea when ultrasound is emitted toward the cornea and on the radiation pressure applied to the cornea. The ultrasonic tonometer described in Patent Document 2 (JP 2009-268651 A) measures the intraocular pressure of the subject eye based on the relationship between characteristics (phase, amplitude) of a reflected wave from the cornea and intraocular pressure.

### SUMMARY

In an ophthalmic apparatus that performs measurement of characteristics of the subject eye using ultrasound, it is necessary to irradiate the subject eye with ultrasound having sufficient output. For example, in order to measure the intraocular pressure of the subject eye using ultrasound, it is necessary to irradiate the subject eye with ultrasound at a very high output.

However, despite having conducted various studies, the present applicant has not been able to achieve irradiation with ultrasound having sufficient output. For example, among ultrasonic actuators, there are actuators that are provided with a flexural vibration portion at an axial distal end portion of a sonotrode that propagates ultrasound in air. Such an ultrasonic actuator provided with a flexural vibration portion may be capable of irradiating the subject eye with ultrasound at a higher output than an ultrasonic actuator without a flexural vibration portion. However, merely adopting an ultrasonic actuator provided with a flexural vibration portion has not enabled realization of irradiation with ultrasound having sufficient output.

Accordingly, a technique is desired that can emit ultrasound to the subject eye with higher efficiency.

A typical object of the present disclosure is to provide an ultrasonic tonometer and an ophthalmic ultrasonic actuator that are capable of emitting ultrasound to the subject eye with high efficiency.

An ultrasonic tonometer according to a typical embodiment of the present disclosure is an ultrasonic tonometer measuring an intraocular pressure of a subject eye using ultrasound. The ultrasonic tonometer includes: an ultrasonic actuator having an ultrasonic element and configured to irradiate the subject eye with ultrasound generated by the ultrasonic element. The ultrasonic actuator includes: a sonotrode propagating ultrasound generated by the ultrasonic element into air; a flexural vibration portion disposed on a distal side of the sonotrode in an axial direction of the ultrasonic actuator, the flexural vibration portion undergoing flexural vibration due to ultrasonic vibration of the sonotrode; and a reflecting portion covering, with a gap with the flexural vibration portion, at least a part of the flexural vibration portion in a circumferential direction, the reflecting portion reflecting the ultrasound generated by the flexural vibration portion and guiding an irradiation direction of the ultrasound of the ultrasonic actuator toward the distal side of the sonotrode in the axial direction.

An ophthalmic ultrasonic actuator according to a typical embodiment of the present disclosure is an ophthalmic ultrasonic actuator for an ophthalmic apparatus that irradiates a subject eye with ultrasound. The ophthalmic ultrasonic actuator includes: an ultrasonic element generating ultrasound; a sonotrode propagating ultrasound generated by the ultrasonic element into air; a flexural vibration portion disposed on a distal side of the sonotrode in an axial direction of the ultrasonic actuator, the flexural vibration portion undergoing flexural vibration due to ultrasonic vibration of the sonotrode; and a reflecting portion covering, with a gap with the flexural vibration portion, at least a part of the flexural vibration portion in a circumferential direction, the reflecting portion reflecting the ultrasound generated by the flexural vibration portion and guiding an irradiation direction of the ultrasound of the ultrasonic actuator toward the distal side of the sonotrode in the axial direction.

A method for measuring an intraocular pressure of a subject eye using ultrasound in a typical embodiment is a method including: generating, with an ultrasonic element of an ultrasonic actuator, ultrasound; propagating, with a sonotrode, ultrasound generated by the ultrasonic element into air; generating, with a flexural vibration portion disposed on a distal side of the sonotrode in an axial direction of the ultrasonic actuator, flexural vibration caused by ultrasonic vibration of the sonotrode; and reflecting, with a reflecting portion covering, with a gap with the flexural vibration portion, at least a part of the flexural vibration portion in a circumferential direction, the ultrasound generated by the flexural vibration portion to guide the ultrasound of the ultrasonic actuator toward the distal side of the sonotrode.

According to the ultrasonic tonometer and the ophthalmic ultrasonic actuator of the present disclosure, ultrasound can be emitted to the subject eye with high efficiency.

The ultrasonic tonometer exemplified in the present disclosure measures intraocular pressure of the subject eye using ultrasound. The ultrasonic tonometer includes the ultrasonic actuator. The ultrasonic actuator has the ultrasonic element and irradiates the subject eye with ultrasound. The ultrasonic actuator of the present disclosure includes the sonotrode, the flexural vibration portion, and the reflecting portion. The sonotrode propagates ultrasound generated by the ultrasonic element in air. The flexural vibration portion is provided on an axial distal side of the sonotrode and flexurally vibrates with ultrasonic vibration of the sonotrode. The reflecting portion, with a gap formed between the reflecting portion and the flexural vibration portion, covers at least a portion of the flexural vibration portion in a circumferential direction so as to reflect ultrasound generated from the flexural vibration portion and guide an irradiation direction of ultrasound by the ultrasonic actuator toward a distal side in the axial direction.

According to the ultrasonic tonometer and the ultrasonic actuator of the present disclosure, even ultrasound that has propagated in a direction other than toward the axial distal side from the flexural vibration portion is reflected by the reflecting portion and is more likely to be guided toward the axial distal side. As a result, ultrasound generated by the ultrasonic actuator can be emitted to the subject eye with high efficiency.

It should be noted that the reflecting portion may cover the entire circumference of the flexural vibration portion in the circumferential direction. In this case, since the ultrasound is more likely to be guided toward the axial distal side over the entire circumference in the circumferential direction, the emission efficiency of the ultrasound to the subject eye is more likely to be further improved. However, the reflecting portion may cover only a part in the circumferential direction of the flexural vibration portion (for example, only above the sonotrode, only below the sonotrode, only laterally, or only above and below the sonotrode when viewed from the subject eye side). Even in this case, the ultrasound that has propagated from the flexural vibration portion to the surroundings is more likely to be appropriately guided toward the subject eye side by the reflecting portion.

When the reflecting portion is provided over the entire circumference in the circumferential direction of the flexural vibration portion, it is more preferable that the gap between the flexural vibration portion and the reflecting portion be provided over the entire circumference in the circumferential direction of the flexural vibration portion. In this case, the ultrasound that has propagated to a rear-end side of the flexural vibration portion is more likely to be guided to the axial distal side through the gap provided over the entire circumference in the circumferential direction. Accordingly, the ultrasound is more likely to be emitted to the subject eye with higher efficiency. However, at a part in the circumferential direction of the flexural vibration portion, the flexural vibration portion and the reflecting portion may be in contact with each other. For example, by providing a mesh-like member between the flexural vibration portion and the reflecting portion, it is possible to reduce the possibility that foreign matter enters the gap. Even in this case, the ultrasound is more likely to be appropriately guided toward the axial distal side by the reflecting portion.

The specific configuration of the flexural vibration portion can be appropriately selected.

For example, the flexural vibration portion and the sonotrode may be formed integrally. In this case, by annularly notching a portion of a body portion of the sonotrode having a columnar external shape (e.g., cylindrical) at a location slightly on an axial proximal side of the distal end, a portion remaining on a distal side of the notched portion may serve as the flexural vibration portion. In this case, the sonotrode provided with the flexural vibration portion is appropriately manufactured from a single member. Note that, when viewed in a cross section including the axis of the sonotrode, a shape of the notched portion may be formed in an arc shape or an elliptical-arc shape. In this case, the ultrasound having a higher output is more likely to be emitted toward the axial distal side. Additionally, the flexural vibration portion and the sonotrode may be separate members. The flexural vibration portion may be fixed to a distal end portion of the sonotrode by a fixing member such as a screw.

Even in this case, when the flexural vibration portion flexurally vibrates, the ultrasound having a high output is more likely to be emitted.

Note that an ultrasonic actuator may be a Langevin transducer. A Langevin transducer may include an ultrasonic element, the sonotrode (also referred to as a horn or a front mass), and a back mass. The ultrasonic element generates the ultrasound. As described above, the sonotrode propagates the ultrasound generated by the ultrasonic element into air. The sonotrode is a mass member disposed on an axial distal side (subject-eye side) of the ultrasonic element. The back mass is a mass member disposed on an axial rear-end side of the ultrasonic element. The sonotrode and the back mass may be clamped together with the ultrasonic element disposed therebetween. Since a Langevin transducer can generate the ultrasound with reduced mechanical and thermal energy losses, it can generate the ultrasound at a high output. However, the reflecting portion exemplified in the present disclosure can be employed in various ones of the ultrasonic actuators provided with the flexural vibration portion. That is, the reflecting portion exemplified in the present disclosure can also be applied to the ultrasonic actuators other than the Langevin transducer.

Although a material of the reflecting portion can be appropriately selected, it is desirable to adopt, as a material of the reflecting portion, a material having as large a difference in acoustic impedance from air as possible. For example, it is possible to use aluminum or a resin as the material of the reflecting portion.

An axial distal end portion of the reflecting portion may protrude further distally than an axial distal end portion of the flexural vibration portion. In this case, at least a part of the ultrasound that has propagated in an obliquely distal direction as viewed from the flexural vibration portion is also reflected by the inner circumferential surface of the reflecting portion and emitted toward the subject eye. Accordingly, the ultrasound generated by the ultrasonic actuator becomes more likely to be emitted toward the subject eye with higher efficiency.

However, the reflecting portion only needs to be provided so as to cover, in the axial direction of the sonotrode, at least a rear-end side from the position of the flexural vibration portion. That is, regardless of whether or not the reflecting portion protrudes further distally than an axial distal end portion of the flexural vibration portion, if it is provided so as to cover a rear-end side from the position of the flexural vibration portion, the ultrasound that has propagated axially rearward from the flexural vibration portion is also appropriately guided more easily toward the axial distal side.

On at least a part of the inner peripheral surface (that is, the surface facing the axis) at the axial distal end portion of the reflecting portion, a first inclined surface that is inclined inward, that is, in a direction toward the axis of the sonotrode and toward the distal side, may be formed. When the first inclined surface is provided, the ultrasound that has passed through the distal opening of the reflecting portion is more likely to converge on the object to be irradiated with ultrasound (in this disclosure, the cornea of the eye to be examined). Accordingly, the ultrasound generated by the ultrasound actuator is more readily emitted to the eye to be examined with higher efficiency. Note that the first inclined surface can also be expressed as an inclined surface inclined so as to face a rear-end side in the axial direction of the sonotrode.

Note that the range, within the inner peripheral surface of the reflecting portion, of an axial distal end portion where the first inclined surface is formed can be set as appropriate. For example, when the axial distal end portion of the reflecting portion protrudes further distally than the axial distal end portion of the flexural vibration portion, the first inclined surface may be formed, in the reflecting portion, at least on the inner peripheral surface on the distal side of the flexural vibration portion. In this case, the ultrasound is more likely to converge further on the object. As one example, in this disclosure, the first inclined surface is formed, in the axial direction of the inner peripheral surface of the reflecting portion, at least within a range from the distal position of the flexural vibration portion to the position of the axially most distal portion of the inner peripheral surface of the reflecting portion. Specifically, in this disclosure, the first inclined surface is formed in a range from the axially most distal portion of the inner peripheral surface of the reflecting portion to the axially most distal portion of a second inclined surface to be described later. Note that, in the reflecting portion, it is not necessary that the entirety of the inner peripheral surface on the distal side of the flexural vibration portion be the first inclined surface. That is, it suffices that the first inclined surface is formed at a position where the ultrasound readily converges on the object.

However, the inner peripheral surface at the axial distal end portion of the reflecting portion may be formed so as to be parallel to the axis of the sonotrode. Even in this case, the ultrasound is appropriately guided more easily toward the axial distal side by the reflecting portion.

The outer peripheral surface at the axial distal end portion of the reflecting portion (that is, a surface facing away from the axis) may be formed as an outer peripheral inclined surface that is inclined in a direction that approaches the axis of the sonotrode toward the distal side (that is, inward). By providing the outer peripheral inclined surface, space around the reflecting portion is more readily secured. Accordingly, installing the ultrasonic actuator including the reflecting portion in an apparatus becomes even easier. For example, even when a plurality of ultrasonic actuators are arranged as densely as possible, installation of the ultrasonic actuators is facilitated.

An annular space of a shape annular about the axis, which is surrounded by the distal end portion of a body portion of the sonotrode that supports the flexural vibration portion, the flexural vibration portion provided at the distal end portion of the body portion, and the inner peripheral surface of the reflecting portion, may be formed. In at least part of a portion of the inner peripheral surface of the reflecting portion that faces the annular space, the second inclined surface, which is inclined in a direction that moves away from the axis of the sonotrode toward the distal side (that is, outward), may be formed. In this case, the ultrasound that has propagated through the annular space is more readily reflected by the second inclined surface toward the axial distal side. That is, the ultrasound that has propagated from the flexural vibration portion toward the axial rear end side is also more readily reflected by the second inclined surface toward the axial distal side. In addition, in the vicinity of the annular space, when two surfaces adjacent to each other at an angle of 90 degrees or less are present, unintended phenomena such as diffraction are likely to occur at a corner portion defined by the two surfaces. As a result, there is a possibility that degradation in the quality of the ultrasound irradiated onto the subject eye will occur. In contrast, by providing the second inclined surface on the inner peripheral surface of the reflecting portion, in the vicinity of the annular space, it is possible to omit or reduce two surfaces that are adjacent to each other at an angle of 90 degrees or less. Accordingly, the ultrasound generated by the ultrasonic actuator is more readily emitted appropriately toward the subject eye. Note that the second inclined surface can also be expressed as an inclined surface that is inclined so as to face the distal side in the axial direction of the sonotrode.

Note that, of the inner peripheral surface of the reflecting portion, the range over which the second inclined surface is formed can be set as appropriate. For example, in the axial direction of the sonotrode, the proximal-side end of the second inclined surface may coincide with the proximal-side end of the annular space. In this case, in the vicinity of the annular space, the presence of two surfaces that are adjacent to each other at an angle of 90 degrees or less is reduced. Accordingly, the ultrasound generated by the ultrasonic actuator is more readily emitted appropriately toward the subject eye.

Additionally, the distal-side end of the second inclined surface may be smoothly connected to the proximal-side end of the first inclined surface described above. In this case, the ultrasound that has propagated through the annular space is more readily guided appropriately toward the distal side.

However, of the inner peripheral surface of the reflecting portion, it is also possible to change the respective ranges over which the first inclined surface and the second inclined surface are formed. For example, in the axial direction of the sonotrode, a surface parallel to the axis of the sonotrode may be formed between the first inclined surface and the second inclined surface. Even in this case, by providing the reflecting portion, the ultrasound is more readily guided appropriately toward the distal side.

When viewed in a cross-section including the axis of the sonotrode, the cross-sectional shape of the second inclined surface formed on the inner peripheral surface of the reflecting portion may be a curved shape inclined in a direction that becomes farther from the axis of the sonotrode toward the distal side. In this case, the ultrasound that has propagated through the annular space is even more efficiently reflected toward the distal side by the second inclined surface.

As one example, in the present disclosure, the cross-sectional shape of the second inclined surface, as viewed in a cross-section including the axis, is formed as a partial circular arc.

As a result, the ultrasound is efficiently reflected toward the distal side. However, the cross-sectional shape of the second inclined surface may be formed as a curved shape other than a partial circular arc (for example, a partial elliptical arc, a quadratic curve, or a sinusoidal curve, etc.). Further, it is also possible to form the cross-sectional shape of the second inclined surface, as viewed in a cross-section including the axis, to be linear. Even in this case, as compared with a case where the second inclined surface is not formed on the inner peripheral surface of the reflecting portion, the ultrasound is more favorably and readily guided toward the distal side.

An annular space of a shape that is annular about the axis, which is surrounded by the distal end portion of the main body portion of the sonotrode that supports the flexural vibration portion, the flexural vibration portion provided at the distal end portion of the main body portion, and the inner peripheral surface of the reflecting portion, may be formed. The ultrasonic tonometer may further include a fixing portion that fixes the position of the reflecting portion with respect to the sonotrode on an axially proximal side of the annular space. On an axially distal side of the fixing portion, the sonotrode and the reflecting portion may be spaced apart over the entire circumference in the circumferential direction. In this case, as compared with a case where the reflecting portion and the sonotrode are in contact on the distal side of the fixing portion, the ultrasonic vibration of the sonotrode is more readily and appropriately performed. Therefore, the ultrasound is more readily and appropriately irradiated to the eye to be examined.

The ultrasonic tonometer may further include a back mass, a flange portion, and the fixing portion. The back mass is disposed on an axially rear end side of the ultrasonic element and clamps the ultrasonic element between itself and the sonotrode. The flange portion is provided on at least one of the sonotrode and the back mass and extends in a direction away from the axis. The fixing portion is provided on the reflecting portion and, by being mounted to the flange portion via a vibration-damping member that attenuates vibration, fixes the position of the reflecting portion with respect to the sonotrode. In this case, since the fixing portion of the reflecting portion is mounted to the flange portion via the vibration-damping member, the ultrasonic vibration of the sonotrode is more readily and appropriately performed. Therefore, the ultrasound is more readily and appropriately irradiated to the eye to be examined.

The reflecting portion, which is mounted to the flange portion via the vibration-damping member, may be mounted to a holding portion that holds the ultrasonic actuator. In this case, as compared with a case where at least one of the sonotrode and the back mass is directly mounted to the holding portion, the ultrasonic vibration of the sonotrode is more readily and appropriately performed. Therefore, the ultrasound is more readily and appropriately irradiated to the eye to be examined.

Note that the flange portion may be provided on the sonotrode rather than on the back mass.

In a Langevin-type transducer, it is necessary to connect wiring to an electrode disposed between the sonotrode and the back mass. By providing the flange portion on the sonotrode, wiring connected to the electrode can be easily passed to the axially rear end side. The complication of the configuration of the ultrasonic actuator is suppressed. However, it is also possible to provide the flange portion on the back mass.

The cross-sectional shape of the inner peripheral surface of the reflecting portion, when viewed in a cross section perpendicular to the axis at an arbitrary position in the axial direction, can be appropriately selected. For example, the cross-sectional shape of the inner peripheral surface of the reflecting portion in a cross section perpendicular to the axis may be circular or elliptical. In this case, since corners are less likely to be formed on the inner peripheral surface of the reflecting portion, a reduction such as diffraction at the corners is less likely to occur. Additionally, when the cross-sectional shape of the inner peripheral surface of the reflecting portion is made circular, the irradiation direction of the ultrasound guided by the reflecting portion is more likely to be stabilized. By making the cross-sectional shape of the inner peripheral surface of the reflecting portion an ellipse according to the space for arranging the ultrasonic actuator, the degree of freedom in arranging the ultrasonic actuator is more readily ensured. However, it is also possible to adopt, for the cross-sectional shape of the inner peripheral surface of the reflecting portion in a cross section perpendicular to the axis, a shape other than a circle or an ellipse (e.g., a polygonal shape or a sector shape).

The ultrasonic actuator may further include, in a state covering the distal opening of the reflecting portion, a protection member (e.g., a mesh member or a slit member) that allows ultrasound to pass from the distal opening toward the distal side in the axial direction. By providing the protection member, a user's finger or foreign matter is less likely to contact components such as a sonotrode inside the reflecting portion. Accordingly, the ultrasonic vibration of the sonotrode is more likely to be performed appropriately.

An ultrasonic tonometer may include a plurality of the ultrasonic actuators. The ultrasonic tonometer may applanate the cornea by irradiating the cornea of the subject eye with ultrasound generated by a plurality of the ultrasonic actuators. In this case, by employing a plurality of the ultrasonic actuators provided with the reflecting portion, ultrasound is irradiated onto the cornea at higher power. As a result, the cornea is more likely to be properly applanated, and thus ultrasonic tonometry is more likely to be performed appropriately.

### BRIEF DESCRIPTION OF THE DRAWINGS

FIG. 1 is a side view showing an external appearance of the ultrasonic tonometer 1.

FIG. 2 is a plan view showing a schematic configuration inside the housing of the ultrasonic tonometer.

FIG. 3 is a diagram showing a schematic configuration of the irradiation unit 100 as viewed from the side of the subject eye E.

FIG. 4 is a block diagram showing a schematic configuration of the control system of the ultrasonic tonometer 1.

FIG. 5 is a perspective view of the ultrasonic actuator 10 of the present embodiment.

FIG. 6 is a cross-sectional view of the ultrasonic actuator 10 in a plane including the axis O.

FIG. 7 is an enlarged cross-sectional view of the region R in FIG. 6.

FIG. 8 is a graph showing the results of a comparative test.

### DESCRIPTION OF THE EMBODIMENT

Hereinafter, one typical embodiment according to the present disclosure will be described with reference to the drawings. An ultrasonic tonometer 1 includes an ultrasonic actuator 10 that emits ultrasound, and measures the intraocular pressure of a subject eye E in a non-contact manner using ultrasound emitted by the ultrasonic actuator 10. For example, the ultrasonic tonometer 1 can measure the intraocular pressure of the subject eye E by optically or acoustically detecting a change in shape or a vibration of the subject eye E associated with irradiation of the subject eye E with ultrasound. As one example, the ultrasonic tonometer 1 continuously irradiates the cornea of the subject eye E with a pulsed wave or a burst wave, and may measure the intraocular pressure of the subject eye E based on output information of ultrasound when the cornea is deformed into a predetermined state (for example, an applanated state or a flattened state). The output information of ultrasound refers to at least one of, for example, sound pressure of the ultrasound, acoustic radiation pressure, irradiation time (for example, elapsed time after a trigger signal to start irradiation of the ultrasound is input), and frequency. In deforming the cornea of the subject eye E, for example, sound pressure of ultrasound, acoustic radiation pressure, or acoustic streaming may be used. Further, the ultrasonic tonometer 1 may measure the intraocular pressure of the subject eye E based on an ultrasonic signal received in association with irradiation of the subject eye E with ultrasound. In this case, as the ultrasonic signal, at least one of amplitude and frequency of the ultrasound can be employed. Note that the ultrasonic actuator 10 can also be used in an ophthalmic apparatus other than the ultrasonic tonometer 1.

### Schematic Configuration

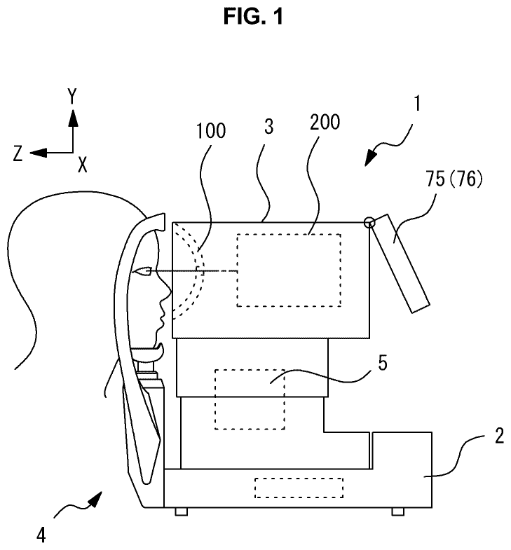

With reference to FIG. 1, a schematic configuration of the ultrasonic tonometer 1 will be described. As shown in FIG. 1, in the present embodiment, the longitudinal direction of the device is defined as the Z direction, the lateral direction as the X direction, and the vertical direction as the Y direction. The ultrasonic tonometer 1 of the present embodiment includes a base 2, a housing 3, a face support portion 4, a drive unit 5, and the like. The base 2 supports the entire apparatus, including the housing 3 of the ultrasonic tonometer 1. The housing 3 is provided with an irradiation unit 100 and an optical system 200, which will be described later, and the like. The housing 3 is further provided with a display unit 75 for displaying various images and an operation unit 76 (for example, a touch panel provided on the display unit 75) for allowing the examiner to input various instructions into the ultrasonic tonometer 1. The face support portion 4 supports the subject's face. The face support portion 4 of the present embodiment is installed on the base 2. However, the face support portion 4 may be installed at a location other than the base 2. The drive unit 5 changes the relative position between the subject eye E, which is supported by the face support portion 4, and the housing 3. The drive unit 5 of the present embodiment changes the relative position between the subject eye E and the housing 3 by moving the housing 3 relative to the base 2.

### Irradiation Unit

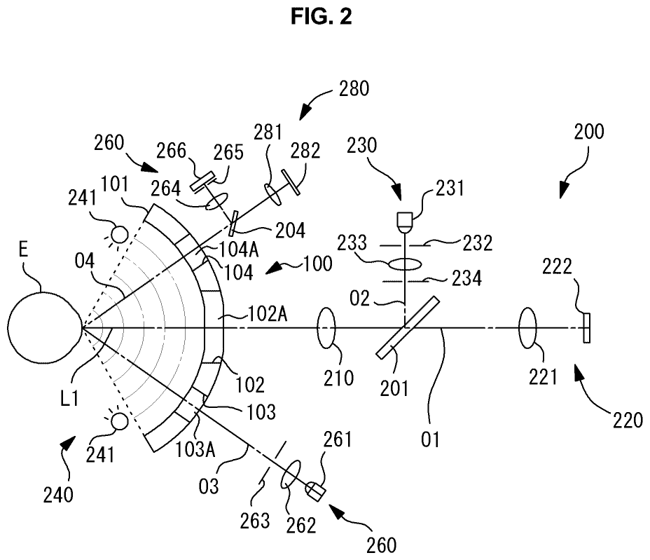

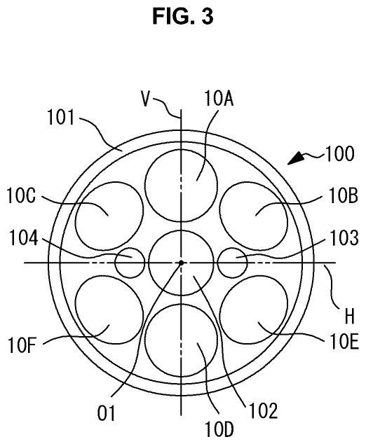

With reference to FIGS. 2 and 3, the configuration of the irradiation unit 100 in the ultrasonic tonometer 1 according to the present embodiment will be described. FIG. 2 is a plan view showing a schematic configuration inside the housing of the ultrasonic tonometer 1 according to the present embodiment. FIG. 3 is a diagram showing a schematic configuration of the irradiation unit 100 as viewed from the subject eye E side. As shown in FIG. 2, the irradiation unit 100 is arranged in front of the subject eye E and irradiates ultrasonic waves onto the subject eye E (the cornea of the subject eye E in the present embodiment). As shown in FIG. 3, a plurality of ultrasonic actuators 10 (10A, 10B, 10C, 10D, 10E, 10F) are arranged in the irradiation unit 100 of the present embodiment. That is, the irradiation unit 100 of the present embodiment can also be referred to as a parametric speaker. In the present embodiment, the ultrasonic tonometer 1 flattens the cornea by irradiating the cornea of the subject eye E with ultrasonic waves generated by a plurality of ultrasonic actuators 10. As a result, since the cornea is more easily properly flattened by the plurality of ultrasonic actuators 10, the tonometry using ultrasonic waves can be more appropriately performed. A detailed description of the ultrasonic actuator 10 will be provided later.

A plurality of ultrasonic actuators 10 are held by the holding unit 101 with their axial end sides (the sides where the ultrasonic waves are emitted) facing the subject eye side. As shown in FIG. 2, the shape of the holding unit 101 on the subject eye E side in the present embodiment is a partial spherical surface. By being held by the holding unit 101, the plurality of ultrasonic actuators 10 are arranged on the spherical surface. As a result, since the acoustic axes of the plurality of ultrasonic actuators 10 each intersect at one point, the ultrasonic waves are more likely to converge at the point where the plurality of acoustic axes intersect. However, the plurality of ultrasonic actuators 10 do not necessarily have to be arranged on the spherical surface and may be arranged on a curved surface such as an ellipsoidal surface, or even on a flat surface. It is also possible to control the phase of the ultrasonic waves irradiated onto the subject eye E by appropriately adjusting the drive waveform and the like of the plurality of ultrasonic actuators 10. By controlling the phase of the ultrasonic waves irradiated onto the subject eye E, at least one of adjustment of the focal position and control of increase or decrease in ultrasonic output may be performed. Further, as an example, although six ultrasonic actuators 10 (10A to 10F) are used in the irradiation unit 100 of the present embodiment, it goes without saying that the number of ultrasonic actuators 10 used is not limited to six. A single ultrasonic actuator 10 may also be used alone in an ophthalmic apparatus such as an ultrasonic tonometer.

As shown in FIGS. 2 and 3, the holding unit 101 is provided with an opening 102 for allowing the optical axis of the optical system 200 to pass through. In the present embodiment, an opening 102 through which the optical axis O1 of the observation system (imaging optical system) 220, which will be described later, passes is provided at the center of the holding unit 101. That is, the optical axis O1 of the observation system 220 is disposed at the opening 102 of the holding unit 101. As described above, since the holding unit 101 that holds the plurality of ultrasonic actuators 10 is provided with the opening 102 for allowing the optical axis O1 of the observation system 220 to pass through, ultrasonic waves with sufficient output are irradiated onto the subject eye E, and the subject eye E is appropriately observed via the observation system 220. Further, in the present embodiment, a first hole 103 through which the light projection optical axis O3 of the deformation detection system 260 (or the Z alignment detection system 280) passes is provided on one of the left and right sides of the holding unit 101, and a second hole 104 through which the light receiving optical axis O4 of the deformation detection system 260 (or the Z alignment detection system 280) passes is provided on the other side. The opening 102 is provided with a light-transmitting member (for example, a transparent plate, lens, or the like) 102A that allows light to pass through while suppressing intrusion of foreign matter into the ultrasonic actuator 10. Similarly, a light-transmitting member 103A is provided in the first hole 103, and a light-transmitting member 104A is provided in the second hole 104.

As shown in FIG. 3, the plurality of ultrasonic actuators 10A to 10F are held by the holding unit 101 so as to surround the periphery of the opening 102 through which the optical axis O1 of the observation system 220 passes. Specifically, in the present embodiment, the plurality of ultrasonic actuators 10A to 10F are arranged so that the sound source areas (the total area of the ultrasonic irradiation surfaces of each of the ultrasonic actuators 10A to 10F) are symmetrical with respect to the upper and lower regions when the holding unit 101 is divided by the horizontal plane H containing the optical axis O1. Moreover, the plurality of ultrasonic actuators 10A to 10F are arranged so that the sound source areas are symmetrical with respect to the left and right regions when the holding unit 101 is divided by the vertical plane V containing the optical axis O1. It is possible to suppress deflection of the acoustic axis L1 of the ultrasonic wave with respect to the optical axis O1, and ultrasonic waves can be more appropriately irradiated onto the subject eye E. It should be noted that the acoustic axis is the central axis of ultrasonic waves irradiated by the irradiation unit 100. The acoustic axis extends in the propagation direction of the ultrasonic wave or in the vibration direction of the irradiation unit 100, and passes through the focal position at which the ultrasonic waves output by the irradiation unit 100 are focused.

As shown in FIG. 3, when the irradiation unit 100 is viewed from the subject eye E side, the shapes of the ultrasonic actuators 10A to 10F (specifically, the shapes inside the reflecting portions 50, as described later) are circular or elliptical. Therefore, it becomes difficult for corners to be formed on the inner peripheral surface of the reflecting portion 50, so that reductions such as diffraction at the corners are less likely to occur. As a result, it becomes easier for the ultrasonic waves to be more appropriately irradiated onto the subject eye E. Specifically, when the irradiation unit 100 is viewed from the subject eye E side, the shapes of the ultrasonic actuators 10A and 10D are circular. Therefore, the ultrasonic irradiation direction by the ultrasonic actuators 10A and 10D becomes more likely to be stable. Further, when the irradiation unit 100 is viewed from the subject eye E side, the shapes of the ultrasonic actuators 10B, 10C, 10E, and 10F are made elliptical in accordance with the installation spaces for the respective ultrasonic actuators 10 in the holding unit 101. As a result, the degree of freedom in arranging the plurality of ultrasonic actuators 10A to 10F is appropriately ensured. However, it is also possible to change the shape inside the reflecting portion 50 of the ultrasonic actuators 10A to 10F.

Assume a case where the relative position in the XYZ directions between the subject eye E and the housing of the ultrasonic tonometer 1 is adjusted (so-called alignment) and the optical axis O1 of the observation system 220 passes through the cornea (hereinafter, referred to as the “alignment completion state”). In the present embodiment, in the alignment completion state, the plurality of the ultrasonic actuators 10A to 10F are held by the holding part 101 so that all of the plurality of the ultrasonic actuators 10A to 10F are accommodated within a range of angles of 48 degrees or less with respect to the optical axis O1, as viewed from the subject eye E (specifically, from the corneal apex of the subject eye E). In this case, the possibility that part of the ultrasonic wave emitted from the irradiation unit 100 toward the subject eye E is blocked by the subject's nose is reduced. Therefore, it becomes easier for the ultrasonic wave to be more appropriately irradiated onto the subject eye E.

### Optical System

With reference to FIG. 2, the configuration of the optical system 200 in the ultrasonic tonometer 1 of the present embodiment will be described. The optical system 200 is used for executing at least one of observation and measurement of the subject eye. The optical system 200 of the present embodiment comprises the objective system 210, the observation system 220, the fixation target projection system 230, the illumination system 240, the deformation detection system 260, the Z alignment detection system 280, the dichroic mirror 201, and the beam splitter 204, among others.

The objective system 210 is an optical system for performing at least one of introduction of light from outside the housing 3 into the optical system 200 and emission of light from the optical system 200 to outside the housing 3. The objective system 210 includes an optical element (for example, at least one of an objective lens and a relay lens).

The illumination system 240 includes the illumination light source 241 and illuminates the subject eye E. The illumination system 240 of the present embodiment illuminates the subject eye E with infrared light by the illumination light source 241, which is an infrared light source. In the present embodiment, a plurality of the illumination light sources 241 are arranged obliquely in front of the subject eye E.

The observation system 220 captures an observation image of the subject eye E (in the present embodiment, an anterior segment image of the subject eye E). Specifically, the observation system 220 of the present embodiment includes the light receiving lens 221 and the light receiving element 222. The observation system 220 of the present embodiment receives a reflected light beam from the subject eye E centered on the optical axis O1. That is, at least part of the light emitted from the illumination light source 241 and reflected by the subject eye E is received by the light receiving element 222. In the present embodiment, the reflected light from the subject eye E passes through the opening 102 of the irradiation unit 100 and is received by the light receiving element 222 via the objective system 210 and the light receiving lens 221. In the present embodiment, the corneal reflection luminous point of the illumination light source 241 received by the light receiving element 222 is used for alignment in vertical, horizontal and lateral directions (XY alignment), for example. In this case, the illumination system 240 and the observation system 220 function as XY alignment detection means. Of course, separately from the illumination system 240, an index projection system may be provided which projects an index for XY alignment onto the subject eye from the optical axis O1. In this case, since the corneal center luminous point appears in the observation image of the observation system 220, XY alignment may be performed based on the corneal center luminous point.

The fixation target projection system 230 projects a fixation target onto the subject eye E. The fixation target projection system 230 of the present embodiment includes the target light source 231, the diaphragm 232, the projection lens 233, the diaphragm 234, and the like. The light from the target light source 231 passes along the optical axis O2 through the diaphragm 232, the projection lens 233, and the diaphragm 234, and is reflected by the dichroic mirror 201. The dichroic mirror 201 aligns the optical axis O2 of the fixation target projection system 230 coaxially with the optical axis O1. The light from the target light source 231 reflected by the dichroic mirror 201 passes along the optical axis O1 through the objective system 210 and is projected onto the subject eye E. When the fixation target of the fixation target projection system 230 is fixated by the subject, the subject's line of sight is stabilized.

The deformation detection system 260 detects deformation of the cornea of the subject eye E. The deformation detection system 260 of the present embodiment includes the light source 261, the projection lens 262, the diaphragm 263, the light receiving lens 264, the diaphragm 265, and the light receiving element 266, and the like. The light from the light source 261 passes along the optical axis O3 through the projection lens 262, the diaphragm 263, and the first aperture 103 of the holding unit 101, and is projected onto the subject eye E. The reflected light emitted from the light source 261 and reflected by the subject eye E passes along the optical axis O4 through the second aperture 104 of the holding unit 101, is reflected by the beam splitter 204, and after passing through the light receiving lens 264 and the diaphragm 265, is received by the light receiving element 266. The deformation detection system 260 detects deformation of the cornea based on the corneal reflected light received by the light receiving element 266.

For example, the deformation detection system 260 may detect the deformation state of the cornea based on the magnitude of the light receiving signal of the light receiving element 266. The deformation detection system 260 may detect the timing when the cornea is in a flattened state as the timing when the amount of received light by the light receiving element 266 becomes the maximum. In this case, the deformation detection system 260 is configured so that the amount of received light becomes maximum when the cornea of the subject eye is in a flattened state. Note that the deformation detection system 260 may be an anterior eye segment cross-sectional image capturing unit such as an OCT or a Scheimpflug camera. Further, the deformation detection system 260 may detect at least one of the deformation amount or deformation speed of the cornea.

The Z alignment detection system 280 detects the relative positional relationship in the Z direction (that is, the alignment state in the Z direction) between the subject eye E and the housing 3. The Z alignment detection system 280 of the present embodiment includes the light receiving lens 281 and the light receiving element 282. The Z alignment detection system 280 of the present embodiment detects the alignment state in the Z direction by detecting reflected light from the cornea of the subject eye E. As an example, the Z alignment detection system 280 of the present embodiment receives the reflected light emitted from the light source 261 and reflected by the cornea of the subject eye E. In this case, the Z alignment detection system 280 may receive the bright spot formed by the light from the light source 261 reflected by the cornea of the subject eye E. That is, in the present embodiment, the light source 261 of the deformation detection system 260 is also used as the light source for the Z alignment detection system 280.

For example, light from the light source 261 reflected by the cornea of the subject eye E passes through the second aperture 104 of the holding unit 101 along the optical axis O4, then passes through the beam splitter 204 and the light receiving lens 281, and is received by the light receiving element 282. When the relative position between the subject eye E and the Z alignment detection system 280 is deviated from the proper position in the Z direction, the light receiving position (for example, the position where the intensity of the received signal is at a maximum) of the light from the light source 261 reflected by the cornea shifts on the light receiving element 282. Therefore, the Z alignment detection system 280 may detect the alignment state based on the light receiving position of the light from the light source 261 on the light receiving element 282.

### Control System

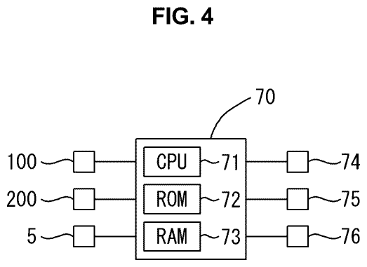

With reference to FIG. 4, the configuration of the control system in the ultrasonic tonometer 1 of the present embodiment will be described. The control unit 70 is responsible for various controls in the ultrasonic tonometer 1 (for example, overall apparatus control, arithmetic processing of measured values, and the like). The control unit 70 may be implemented, for example, by a general CPU (Central Processing Unit) 71, a ROM 72, a RAM 73, and the like. Various programs and initial values for controlling operation of the ultrasonic tonometer 1 are stored in the ROM 72. The RAM 73 temporarily stores various information. Note that the control unit 70 may be configured as a single control unit or as multiple control units (that is, a plurality of processors). The control unit 70 is connected, for example, to the drive unit 5, the storage unit 74, the display unit 75, the operation unit 76, the irradiation unit 100, the optical system 200, and the like.

### Ultrasonic Actuator

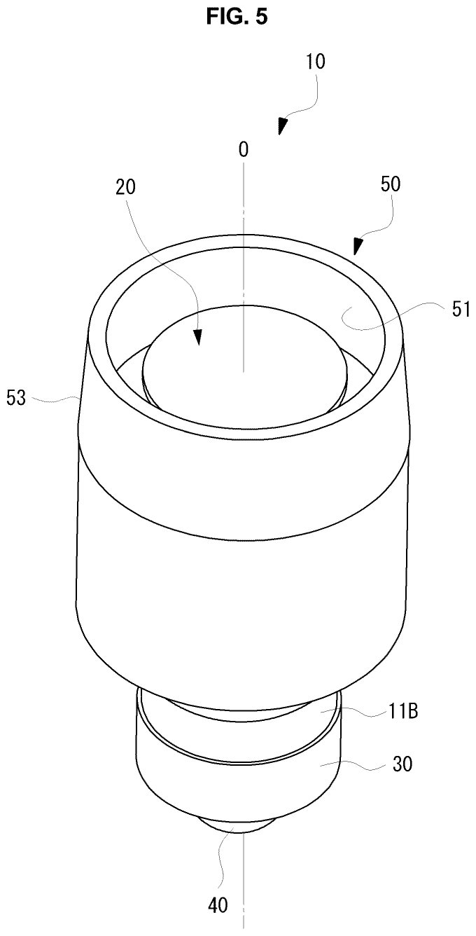

With reference to FIGS. 5 to 7, the ultrasonic actuators 10 (10A to 10F) of the present embodiment will be described in detail. The ultrasonic actuators used in the ultrasonic tonometer 1 of the present embodiment are so-called Langevin-type transducers (also referred to as “bolt-clamped Langevin-type transducers”). In the Langevin-type transducer, the ultrasonic elements 11 (11A, 11B) are compressed by two mass members (the sonotrode 20 and the back mass 30), whereby ultrasonic waves are generated at high efficiency with reduced mechanical and thermal energy losses. Hereinafter, of the six ultrasonic actuators 10A to 10F used in the ultrasonic tonometer 1, one ultrasonic actuator 10A will be described by way of example. It should be noted that the other ultrasonic actuators 10B to 10F may be exactly identical to the ultrasonic actuator 10A described below, or may differ slightly in the shapes of some members or the like. Hereinafter, the axis of the ultrasonic actuator 10 (which is also the axis of the sonotrode 20 of the ultrasonic actuator 10) is denoted by O. Ultrasonic waves are emitted toward the distal end side of the axis O (the upper side in FIGS. 5 to 7).

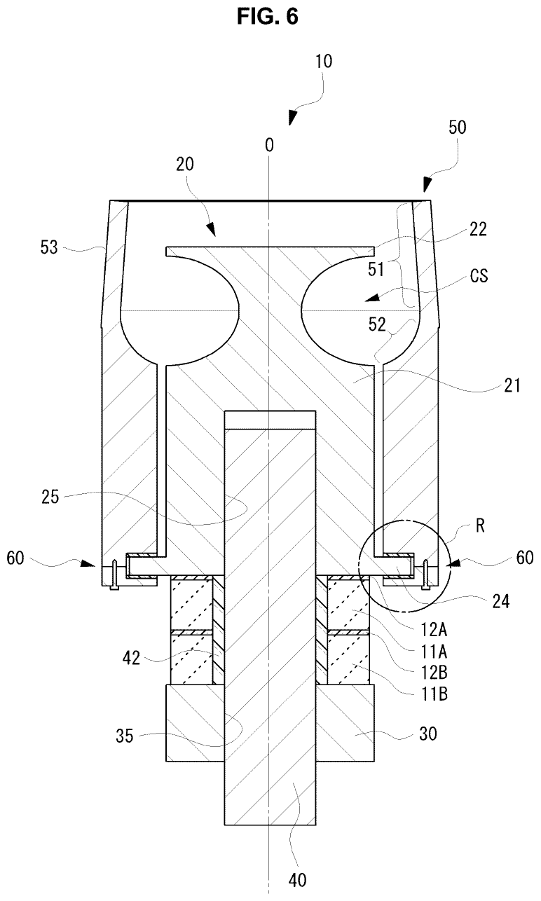

As shown in FIG. 6, the ultrasonic actuator 10 of the present embodiment includes the ultrasonic elements 11 (11A, 11B), the electrodes 12 (12A, 12B), the sonotrode 20, the back mass 30, the bolt 40, and a reflector 50. The ultrasonic element 11 generates ultrasonic waves. For example, a voltage-driven element (such as a piezoelectric ceramic) or a magnetostrictive element may be used as the ultrasonic element 11. The shape of the ultrasonic element 11 of the present embodiment is annular (ring-shaped). In the present embodiment, a plurality of ultrasonic elements, namely the ultrasonic elements 11A and 11B (two in the present embodiment), are used in a stacked state. The electrodes 12 (12A, 12B) are connected to respective ones of the plurality of ultrasonic elements 11A and 11B. The shape of the electrode 12 of the present embodiment is also annular (ring-shaped). In the present embodiment, the electrode 12A is connected to the distal end side of the ultrasonic element 11A, and the electrode 12B is connected to the distal end side of the ultrasonic element 11B.

The sonotrode 20 and the back mass 40 (collectively also referred to as “mass members”) sandwich the ultrasonic element 11 therebetween, thereby increasing the tensile strength of the ultrasonic element 11 and improving vibration resistance. As a result, high-output ultrasonic waves are more readily generated. Metal blocks or the like may be used as the mass members.

The sonotrode (also referred to as a horn or front mass) 20 is a mass member that is disposed on a distal end side in the direction of the axis O of the ultrasonic element 11 (that is, on the eye under examination E side). The sonotrode 20 propagates the ultrasonic waves generated by the ultrasonic element 11 into air. On a distal end side in the direction of the axis O of the sonotrode 20 (i.e., at a position distal to the sonotrode body 21), a flexural vibration portion 22 is provided. The flexural vibration portion 22 undergoes flexural vibration in association with ultrasonic vibration of the sonotrode 20. As a result, ultrasonic waves are more readily irradiated at higher output as compared with a case where the flexural vibration portion 22 is not provided. Note that, in the present embodiment, the flexural vibration portion 22 and the sonotrode body 21 are formed integrally. Specifically, in the present embodiment, among portions of the sonotrode body 21 having an outer shape that is columnar (cylindrical in this embodiment), a portion slightly proximal to a distal end in the direction of the axis O is notched annularly, whereby a portion remaining on the distal side of the notched portion is formed as the flexural vibration portion 22. Accordingly, the sonotrode 20 provided with the flexural vibration portion 22 is suitably manufactured from a single member. Further, in the present embodiment, when the sonotrode 20 is viewed in a cross section including the axis O, a shape of a notched portion formed in the sonotrode body 21 is formed along an arc (which may be an elliptical arc). As a result, ultrasonic waves of higher output are more readily irradiated toward the distal end side in the direction of the axis O.

However, the specific configurations of the sonotrode and the flexural vibration portion may be changed. For example, the flexural vibration portion and the sonotrode may be separate members. In this case, the flexural vibration portion may be fixed to a distal end portion of the sonotrode by a fastening member such as a screw. Even in this case, by the flexural vibration portion undergoing flexural vibration, ultrasonic waves of high output are more readily irradiated.

In the vicinity of a rear end portion in the direction of the axis O of the sonotrode 20, a flange portion 24 that extends in a direction away from the axis O is provided. The flange portion 24 is used to mount, to the ultrasonic tonometer 1, the ultrasonic element 11, the electrode 12, the sonotrode 20, the flexural vibration portion 22, a back mass 30, and a bolt 50 of the ultrasonic actuator 10. Although details will be described later, in this embodiment, a fixing portion 60 of a reflecting portion 50 is mounted to the flange portion 24, and further the reflecting portion 50 is held by a holding portion 101 (see FIGS. 2 and 3), whereby the ultrasonic actuator 10 is mounted to the ultrasonic tonometer 1. Further, in the vicinity of the axis O of the sonotrode 20, a screw hole 25 is formed so as to extend from a rear end portion to a central portion in the direction of the axis O. A bolt 40 is threaded into the screw hole 25.

The back mass 30 is a mass member disposed on a rear end side in the direction of the axis O of the ultrasonic element 11. The back mass 30 clamps the ultrasonic element 11 between itself and the sonotrode 20. In the present embodiment, the shape of the back mass 30 is substantially cylindrical. In the vicinity of the axis O of the back mass 30, a screw hole 35 is formed that extends through in the direction of the axis O from a distal end to a rear end.

The shape of the bolt 40 is substantially columnar or substantially cylindrical. A helical thread is formed on an outer peripheral portion of the bolt 40. The thread of the bolt 40 is threadedly engaged with the screw hole 25 of the sonotrode 20 and the screw hole 35 of the back mass 30. With the ultrasonic element 11 disposed between the sonotrode 20 and the back mass 30, the bolt 40 is threadedly engaged with the screw hole 35 of the back mass 30 and the screw hole 25 of the sonotrode 20. Accordingly, the sonotrode 20 and the back mass 30 are tightened in a direction approaching each other. As a result, the ultrasonic element 11 is strongly clamped by the sonotrode 20 and the back mass 30.

The ultrasonic actuator 10 includes an insulating member 42. The insulating member 42, by being disposed between the inner peripheral surfaces of the ring-shaped ultrasonic elements 11A and 11B and the electrodes 12A and 12B and the outer peripheral surface of the bolt 40, prevents electrical leakage to the bolt 40.

The reflecting portion 50, with a gap formed between itself and the flexural vibration portion 22, covers at least a part (in the present embodiment, the entire circumference) of the circumferential direction of the flexural vibration portion 22 with reference to the axis O. The reflecting portion 50 reflects at least a part of ultrasonic waves generated from the flexural vibration portion 22 and guides the irradiation direction of ultrasonic waves by the ultrasonic actuator 10 toward a distal end side in the direction of the axis O. Ultrasonic waves generated from the flexural vibration portion 22 propagate not only toward the distal end side of the axis O but also in directions away from the axis O or the like. In contrast, in the ultrasonic actuator 10 of the present embodiment, ultrasonic waves that have propagated from the flexural vibration portion 22 in directions different from the distal end side in the direction of the axis O are also reflected by the reflecting portion 50 and are more readily guided toward the distal end side in the direction of the axis O. As a result, ultrasonic waves generated by the ultrasonic actuator 10 are more readily irradiated onto the subject eye E with high efficiency. In the present embodiment, as a material of the reflecting portion 50, aluminum or a resin or the like having a large difference in acoustic impedance with air is adopted.

In the present embodiment, the reflecting portion 50 covers the entire circumference in the circumferential direction of the flexural vibration portion 22. Accordingly, since ultrasonic waves are more readily guided toward the distal end side in the direction of the axis O over the entire circumference in the circumferential direction, the emission efficiency of ultrasonic waves toward the subject eye E is more likely to be further improved. Further, in the present embodiment, the clearance between the flexural vibration portion 22 and the reflecting portion 50 is provided over the entire circumference in the circumferential direction of the flexural vibration portion 22. Accordingly, ultrasonic waves that have propagated to the rear end side of the flexural vibration portion 22 are more readily guided toward the distal end side through the clearance provided over the entire circumference in the circumferential direction.

In the present embodiment, the distal end portion in the direction of the axis O of the reflecting portion 50 (the upper end portion in FIGS. 5 and 6) protrudes further toward the distal end side than the distal end portion in the direction of the axis O of the flexural vibration portion 22. As a result, at least a part of ultrasonic waves that have propagated in an oblique distal end direction as viewed from the flexural vibration portion 22 are also reflected by the inner peripheral surface of the reflecting portion 50 (that is, a surface facing the axis O on the inner side) and emitted toward the subject eye E. Accordingly, ultrasonic waves generated by the ultrasonic actuator 10 are more readily emitted toward the subject eye E with higher efficiency.

In the reflecting portion 50 of the present embodiment, a first inclined surface 51 that is inclined inward in a direction approaching the axis O from its proximal end to its distal end is formed on at least a part of the inner peripheral surface at the distal end portion in the direction of the axis O (that is, a surface facing the axis O). By providing the first inclined surface 51, ultrasonic waves that have passed through the distal opening of the reflecting portion are more readily focused on the object to be irradiated with ultrasonic waves (in the present disclosure, the cornea of the subject eye E). Accordingly, ultrasonic waves generated by the ultrasonic actuator 10 are more readily emitted toward the subject eye E with higher efficiency. Note that the first inclined surface 51 may also be described as an inclined surface that is inclined to face toward a rear end side in the direction of the axis O.

In the reflecting portion 50 of the present embodiment, the first inclined surface 51 is formed at least on the inner peripheral surface on the distal side in the direction of the axis O with respect to the flexural vibration portion 22. As a result, the ultrasonic waves are even more likely to be focused on the object. As one example, in the present embodiment, of the inner peripheral surface of the reflecting portion 50 in the direction of the axis O, at least a range from the distal end position of the flexural vibration portion 22 to the position of the foremost end portion of the inner peripheral surface of the reflecting portion 50 is formed as the first inclined surface 51. More specifically, in the present embodiment, the first inclined surface 51 is formed in a range from the foremost end portion of the inner peripheral surface of the reflecting portion 50 to the foremost end portion of a second inclined surface 52 to be described later. However, it is not necessary that all of the inner peripheral surface on the distal side of the flexural vibration portion 22 in the reflecting portion 50 be the first inclined surface 51. That is, it suffices that the first inclined surface 51 be formed at a position where the ultrasonic waves are more readily focused on the object.

Additionally, in the present embodiment, on the outer peripheral surface at the distal end portion in the direction of the axis O of the reflecting portion 50 (that is, a surface oriented away from the axis O), an outer peripheral inclined surface 53 that is inclined inward in a direction approaching the axis O as it goes toward the distal end side is formed. By providing the outer peripheral inclined surface 53, space around the reflecting portion 50 is more easily secured. Accordingly, installing the ultrasonic actuator 10 provided with the reflecting portion 50 in an apparatus is further facilitated. For example, as shown in FIG. 3, even when a plurality of the ultrasonic actuators 10 are arranged as densely as possible, installation of the ultrasonic actuators 10 is facilitated.

In the present embodiment, an annular space CS, which is annular in shape about the axis O and is surrounded by a portion on the distal side of the sonotrode main body portion 21, the flexural vibration portion 22 provided at a distal end of the sonotrode main body portion 21, and the inner peripheral surface of the reflecting portion 50, is formed. If the reflecting portion 50 is not provided, ultrasonic waves that have propagated through the annular space CS often escape to the outside in a direction away from the axis O. In contrast, in the present embodiment, among the inner peripheral surface of the reflecting portion 50, a second inclined surface 52 inclined in a direction away from the axis O (i.e., outward) from its proximal end to its distal end is formed at least on a part of a portion facing the annular space CS. Accordingly, ultrasonic waves that have propagated through the annular space CS are more readily reflected by the second inclined surface 52 toward the distal side in the direction of the axis O. That is, ultrasonic waves that have propagated from the flexural vibration portion 22 toward the proximal side in the direction of the axis O are also more readily reflected by the second inclined surface 52 toward the distal side in the direction of the axis O. Further, in the vicinity of the annular space CS, when two surfaces that adjoin each other at an angle of 90 degrees or less are present, undesired phenomena such as diffraction tend to occur at a corner portion enclosed by the two surfaces. As a result, there is a possibility that phenomena such as a deterioration in the quality of the ultrasonic waves irradiated to the subject eye E will occur. In contrast, by providing the second inclined surface 52 on the inner peripheral surface of the reflecting portion 50, it is possible, in the vicinity of the annular space CS, to omit or reduce two surfaces that adjoin each other at an angle of 90 degrees or less. Accordingly, the ultrasonic waves generated by the ultrasonic actuator 10 are more readily and appropriately emitted toward the subject eye E. It should be noted that the second inclined surface 52 may also be expressed as an inclined surface inclined so as to face the distal side in the direction of the axis O.

The second inclined surface 52 of the present embodiment will be described in further detail.

In the present embodiment, in the direction of the axis O, a proximal-side end of the second inclined surface 52 coincides with a proximal-side end of the annular space CS. That is, within the sonotrode 20, a proximal-side end of an annularly cut-out portion located slightly on the proximal side of the foremost end portion and the proximal-side end of the second inclined surface 52 coincide with each other in the direction of the axis O. Accordingly, in the vicinity of the annular space CS, the presence of two surfaces that adjoin each other at an angle of 90 degrees or less is reduced. Accordingly, the ultrasonic waves generated by the ultrasonic actuator 10 are more readily and appropriately emitted toward the subject eye E. Further, a distal-side end of the second inclined surface 52 is smoothly connected to a proximal-side end of the first inclined surface 51 described above. Accordingly, ultrasonic waves that have propagated through the annular space CS are more readily and appropriately guided toward the distal side.

Further, as shown in FIG. 6, when viewed in a cross section including the axis O, the cross-sectional shape of the second inclined surface 52 formed on the inner peripheral surface of the reflecting portion 50 is a curved shape inclined in a direction away from the axis O from the proximal end to the distal end of the second inclined surface 52. In this case, the ultrasonic waves that have propagated through the annular space CS are more efficiently reflected toward the distal side by the second inclined surface 52. As an example, in the present embodiment, the cross-sectional shape of the second inclined surface 52, when viewed in a cross section including the axis O, is formed in a partial arc shape. As a result, the ultrasonic waves are efficiently reflected toward the distal side. However, the cross-sectional shape of the second inclined surface 52 may be formed as a curved shape other than the partial arc shape (for example, a partial elliptical arc shape, a quadratic curve shape, or a sinusoidal curve shape). Further, the cross-sectional shape of the second inclined surface, when viewed in a cross section including the axis, can also be formed to be linear.

As described above, on the proximal side in the axis O direction relative to the annular space CS, the fixed portion 60 for fixing the position of the reflecting portion 50 relative to the sonotrode 20 is provided. In the present embodiment, on the distal side in the axis O direction relative to the fixed portion 60, the sonotrode 20 and the reflecting portion 50 are spaced apart from each other. Accordingly, as compared with a case where the reflecting portion 50 and the sonotrode 20 are in contact on the distal side relative to the fixed portion 60, the ultrasonic vibration of the sonotrode 20 is more readily and appropriately performed. Accordingly, the ultrasonic waves are more readily and appropriately emitted toward the subject eye E.

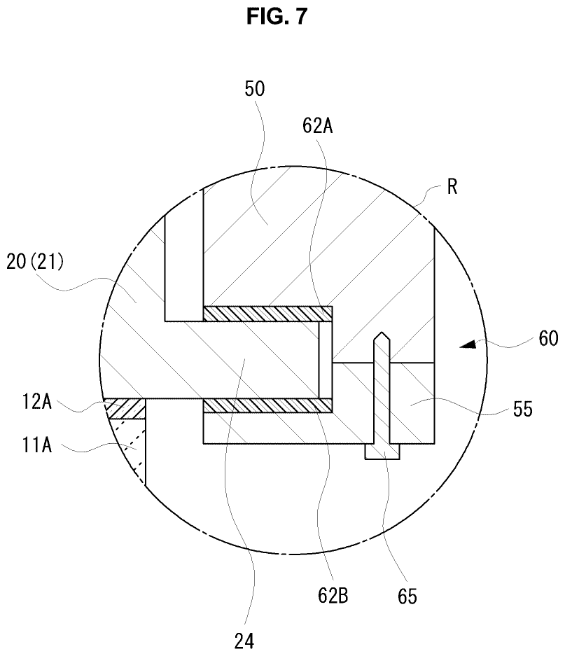

With reference to FIG. 7, a configuration for fixing the position of the reflecting portion 50 relative to the sonotrode 20 will be described. As shown in FIG. 7, in the vicinity of the rear end portion of the sonotrode 20 in the axis O direction, the flange portion 24 that extends in a direction away from the axis O is provided. Further, in a part of the inside of the reflecting portion 50 (in the present embodiment, the inside on the proximal side of the reflecting portion 50), the fixed portion 60 that fixes the position of the reflecting portion 50 relative to the sonotrode 20 is provided. The fixed portion 60 provided on the reflecting portion 50 is mounted to the flange portion 24 via the anti-vibration members 62A and 62B, whereby the position of the reflecting portion 50 relative to the sonotrode 20 is fixed. Accordingly, as compared with a case where the fixed portion 60 is mounted to the sonotrode 20 (the flange portion 24 in the present embodiment) without interposition of the anti-vibration members 62A and 62B, the ultrasonic vibration of the sonotrode 20 is more readily and appropriately performed.

In the example shown in FIG. 7, an annular (ring-shaped) anti-vibration member 62A is provided between the distal-side surface of the flange portion 24 and the reflecting portion 50. Further, an anti-vibration member 62B is provided between the rear-end-side surface of the flange portion 24 and the fixing piece 55 of the reflecting portion 50. As an example, the fixing piece 55 and the anti-vibration member 62B of the present embodiment are formed in an annular (ring-shaped) form. However, it is also possible to change the shapes of the fixing piece 55 and the anti-vibration member 62B. With the flange portion 24, the anti-vibration members 62A and 62B, the reflecting portion 50, and the fixing piece 55 assembled in appropriate positions, the fixing piece 55 is fixed to the rear-end-side surface of the reflecting portion 50 by a screw 65. As a result, the anti-vibration member 62A, the flange portion 24, and the anti-vibration member 62B are sandwiched between the rear-end-side surface of the reflecting portion 50 and the distal-side surface of the fixing piece 55, whereby the position of the reflecting portion 50 relative to the sonotrode 20 is fixed. Note that the anti-vibration members 62A and 62 may be elastic members such as rubber, and anti-vibration grease or the like may be used. However, the fixing method shown in FIG. 7 is merely an example, and it is also possible to change the shapes and the like of the respective components.

As shown in FIG. 7, in the present embodiment, the flange portion 24 is provided on the sonotrode 20 rather than on the back mass 30. As described above, in the ultrasonic actuator 10, it is necessary to connect the wiring to the electrodes 12A and 12B that are disposed between the sonotrode 20 and the back mass 30. By providing the flange portion 24 on the sonotrode 20, the wiring connected to the electrodes 12A and 12B can readily be routed toward the rear-end side in the axis O direction. Accordingly, an increase in complexity of the configuration of the ultrasonic actuator 10 is suppressed. However, it is also possible to provide a flange portion on the back mass 30 and mount the fixed portion of the reflecting portion 50 to the flange portion of the back mass 30.

In the present embodiment, the reflecting portion 50, which is mounted to the flange portion via a vibration-damping member, is mounted to the holding portion 101 (see FIGS. 2 and 3) that holds the ultrasonic actuator 10. Accordingly, as compared with a case in which at least one of the sonotrode 20 and the back mass 30 is directly mounted to the holding portion 101, the ultrasonic vibration of the sonotrode 20 is more readily carried out appropriately. Accordingly, ultrasound is more readily irradiated appropriately onto the eye under examination E.

The cross-sectional shape of the inner peripheral surface of the reflecting portion 50, as viewed in a cross section perpendicular to the axis O at an arbitrary position in the axis O direction, will be described. As described with reference to FIG. 3, in the present embodiment, the cross-sectional shape of the inner peripheral surface of the reflecting portion 50 in a cross section perpendicular to the axis O is circular or elliptical. In this case, corner portions are less likely to occur on the inner peripheral surface of the reflecting portion 50, so that a decrease in diffraction and the like at corner portions is less likely to occur. Moreover, when the cross-sectional shape of the inner peripheral surface of the reflecting portion 50 is circular, the irradiation direction of ultrasound guided by the reflecting portion 50 is more readily stabilized. By adopting, for the cross-sectional shape of the inner peripheral surface of the reflecting portion 50, an elliptical shape corresponding to the space in which the ultrasonic actuator 10 is to be disposed, the degree of freedom in disposition of the ultrasonic actuator 10 is more readily ensured.

Incidentally, although not shown, the ultrasonic actuator 10 may further include a protective member that, while covering the distal opening of the reflecting portion 50, allows ultrasound to pass from the distal opening toward the distal side in the axis O direction (for example, a mesh-like member or a slit-like member). By providing the protective member, it becomes difficult for a user's finger or foreign matter to contact the sonotrode 20 or the like inside the reflecting portion 50. Accordingly, the ultrasonic vibration of the sonotrode 20 is more readily carried out appropriately.

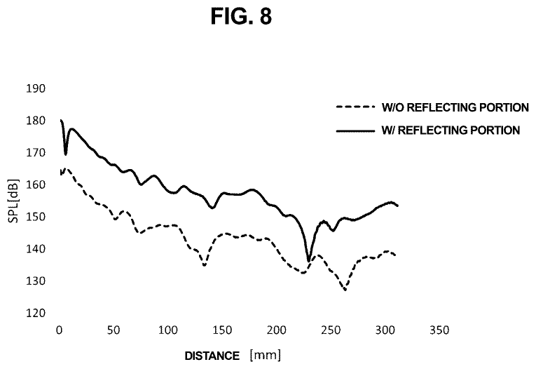

With reference to FIG. 8, the results of a comparative test to confirm the effect of using the reflecting portion 50 will be described. In this test, using each of the ultrasonic actuator 10 equipped with the reflecting portion 50 of the above embodiment (‘with reflecting portion’ in FIG. 8) and the ultrasonic actuator 10 with only the reflecting portion 50 removed (‘without reflecting portion’ in FIG. 8), the sound pressure level was measured while varying the distance from the distal end of the ultrasonic actuator in the direction along the axis O. All conditions other than the presence or absence of the reflecting portion 50 are the same. As shown in FIG. 8, the sound pressure level in the case of ‘with reflecting portion’ was higher than that in the case of ‘without reflecting portion’, irrespective of the distance from the distal end. From the above results, it can be confirmed that, by using the reflecting portion 50, the output of ultrasound irradiated toward the distal direction of the axis O is increased.

The technology disclosed in the above embodiment is merely an example. Accordingly, the technology exemplified in the above embodiment can also be modified. For example, in the above embodiment, by mounting the fixing portion 60 of the reflecting portion 50 on the flange portion 24 having a shape continuous in the circumferential direction at an outer peripheral portion of the mass member (in the present embodiment, the sonotrode 20), the position of the reflecting portion 50 relative to the sonotrode 20 is fixed. However, it is also possible to change the method of fixing the position of the reflecting portion 50 relative to the sonotrode 20. For example, a plurality of fixing members such as projections may be formed at predetermined intervals on an outer peripheral portion of the mass member (at least one of the sonotrode 20 and the back mass 30), and by sandwiching each of the fixing members in the direction of the axis O, the position of the reflecting portion 50 relative to the sonotrode 20 may be fixed. In this case, as in the above embodiment, it is desirable that a vibration-damping member be used between the fixing members and the reflecting portion 50.

1. An ultrasonic tonometer measuring an intraocular pressure of a subject eye using ultrasound, the ultrasonic tonometer comprising:
an ultrasonic actuator having an ultrasonic element and configured to irradiate the subject eye with ultrasound generated by the ultrasonic element, wherein
the ultrasonic actuator includes:
a sonotrode propagating ultrasound generated by the ultrasonic element into air;
a flexural vibration portion disposed on a distal side of the sonotrode in an axial direction of the ultrasonic actuator, the flexural vibration portion undergoing flexural vibration due to ultrasonic vibration of the sonotrode; and
a reflecting portion covering, with a gap with the flexural vibration portion, at least a part of the flexural vibration portion in a circumferential direction, the reflecting portion reflecting the ultrasound generated by the flexural vibration portion and guiding an irradiation direction of the ultrasound of the ultrasonic actuator toward the distal side of the sonotrode in the axial direction.

2. The ultrasonic tonometer according to claim 1, wherein
a distal end of the reflecting portion in the axial direction further protrudes in the axial direction than a distal end of the flexural vibration portion in the axial direction.

3. The ultrasonic tonometer according to claim 1, wherein
a first inclined surface is formed on at least a portion of an inner circumferential surface of a distal end of the reflecting portion, and
the first inclined surface is inclined toward the sonotrode from a proximal end to a distal end of the first inclined surface.

4. The ultrasonic tonometer according to claim 1, wherein
the sonotrode includes a body portion that supports the flexural vibration portion at a distal end of the body portion,
an annular space around an axis of the ultrasonic actuator is defined by the distal end of the body portion of the sonotrode, the flexural vibration portion, and an inner circumferential surface of the reflecting portion,
a second inclined surface is formed on at least a portion of the inner circumferential surface of the reflecting portion that faces the annular space, and
the second inclined surface is inclined in a direction away from the sonotrode from a proximal end to a distal end of the second inclined surface.

5. The ultrasonic tonometer according to claim 4, wherein
in a cross-sectional view including the axis of the ultrasonic actuator, a cross-sectional shape of the second inclined surface is curved in a direction away from the axis of the ultrasonic actuator from the proximal end to the distal end of the second inclined surface.

6. The ultrasonic tonometer according to claim 1, wherein
the sonotrode includes a body portion that supports the flexural vibration portion at a distal end of the body portion,
an annular space around an axis of the ultrasonic actuator is defined by the distal end portion of the body portion of the sonotrode, the flexural vibration portion, and an inner circumferential surface of the reflecting portion,
the ultrasonic tonometer further comprises a fixing portion that fixes, at a proximal side of the reflecting portion in the axial direction, the reflecting portion relative to the sonotrode,
the annular space is located between a distal side and the proximal side of the reflecting portion, and
the distal side of the sonotrode and the distal side of the reflecting portion are spaced away from each other.

7. The ultrasonic tonometer according to claim 1, further comprising:
a back mass arranged on a proximal side of the ultrasonic element in the axial direction, the back mass sandwiching the ultrasonic element together with the sonotrode,
a flange portion disposed in at least one of the sonotrode and the back mass, the flange portion protruding from the at least one of the sonotrode and the back mass in a direction away from an axis of the ultrasonic actuator, and
a fixing portion disposed in the reflecting portion, the fixing portion mounted to the flange portion via a vibration-damping member that attenuates vibration and fixing the reflecting portion relative to the sonotrode.

8. The ultrasonic tonometer according to claim 7, wherein
the reflecting portion mounted to the flange portion via the vibration-damping member is further mounted to a holding portion that holds the ultrasonic actuator.

9. The ultrasonic tonometer according to claim 1, wherein
the ultrasonic actuator includes a plurality of ultrasonic actuators, and
a cornea is applanated by irradiating the cornea of the subject eye with ultrasound generated by the plurality of the ultrasonic actuators.

10. An ophthalmic ultrasonic actuator for an ophthalmic apparatus that irradiates a subject eye with ultrasound, comprising:
an ultrasonic element generating ultrasound;
a sonotrode propagating ultrasound generated by the ultrasonic element into air;
a flexural vibration portion disposed on a distal side of the sonotrode in an axial direction of the ultrasonic actuator, the flexural vibration portion undergoing flexural vibration due to ultrasonic vibration of the sonotrode; and
a reflecting portion covering, with a gap with the flexural vibration portion, at least a part of the flexural vibration portion in a circumferential direction, the reflecting portion reflecting the ultrasound generated by the flexural vibration portion and guiding an irradiation direction of the ultrasound of the ultrasonic actuator toward the distal side of the sonotrode in the axial direction.

11. A method for measuring an intraocular pressure of a subject eye using ultrasound, comprising:
generating, with an ultrasonic element of an ultrasonic actuator, ultrasound;
propagating, with a sonotrade, ultrasound generated by the ultrasonic element into air;
generating, with a flexural vibration portion disposed on a distal side of the sonotrode in an axial direction of the ultrasonic actuator, flexural vibration caused by ultrasonic vibration of the sonotrode; and
reflecting, with a reflecting portion covering, with a gap with the flexural vibration portion, at least a part of the flexural vibration portion in a circumferential direction, the ultrasound generated by the flexural vibration portion to guide the ultrasound of the ultrasonic actuator toward the distal side of the sonotrode.
